(19) United States
(12) Patent Application Publication
(10) Pub. No.: US 2015/0057721 A1
(43) Pub. Date: Feb. 26, 2015

(54) LEADLESS PACEMAKER WITH IMPROVED CONDUCTED COMMUNICATION

(71) Applicant: Cardiac Pacemakers, Inc., St. Paul, MN (US)

(72) Inventors: Jeffrey E. Stahmann, Ramsey, MN (US); William J. Linder, Golden Valley, MN (US); Keith R. Maile, New Brighton, MN (US)

(21) Appl. No.: 14/444,123

(22) Filed: Jul. 28, 2014

Related U.S. Application Data
(60) Provisional application No. 61/869,190, filed on Aug. 23, 2013.

(51) Int. Cl. A61N 1/375; A61N 1/39; A61N 1/365; A61N 1/372; A61N 1/05

(52) U.S. Cl. CPC A61N 1/3756; A61N 1/37223; A61N 1/0573; A61N 1/365; A61N 1/39
USPC 607/60; 29/825

(57) ABSTRACT

A leadless implantable medical device can include a hermetically scaled housing including a cylindrical body, a first surface at a first capped end of the cylindrical body, and a second surface at a second capped end of the cylindrical body. A first electrode can be located at the first capped end and a second electrode can be located on the second surface. The first and second electrodes include conductive portions configured for contacting one or both of tissue and fluid, and wherein the cylindrical body includes a length and the conductive portions of the first and second electrodes are separated substantially by the length of the cylindrical body. The device example also includes a therapy circuit configured to deliver electrical cardiac stimulating energy using the first and second electrodes, and a telemetry circuit configured to communicate with a second separate device.

### CLAIM OF PRIORITY

This application claims the benefit of priority under 35 U.S.C. §119(e) of U.S. Provisional Patent Application Ser. No. 61/869,190, tiled on Aug. 23, 2013, which is herein incorporated by reference in its entirety.

### BACKGROUND

Implantable medical devices can include cardiac function management (CFM) devices such as implantable pacemakers, implantable cardioverter defibrillators (ICDs), cardiac resynchronization therapy devices (CRTs), and devices that include a combination of such capabilities. The devices can be used to treat patients or subjects using electrical or other therapy or to aid a physician or caregiver in patient diagnosis through internal monitoring of a patient's condition. The devices may include one or more electrodes in communication with one or more sense amplifiers to monitor electrical heart activity within a patient, and often include one or more sensors to monitor one or more other internal patient parameters.

Implantable medical devices typically include one or more implantable leads that can be positioned to contact the endocardium within one or more heart chambers or positioned to contact the epicardium. The leads include one or more electrodes to deliver electrical stimulation therapy or to sense intrinsic cardiac activity. The leads can be a source of potential device malfunction due to mechanical or electrical failure. An implantable device also typically includes an electronics unit within a hermetically sealed housing. The interface between the leads and the electronics unit can also be a source of potential device malfunction.

A leadless approach for endocardial pacing can address some of the challenges associated with implantable leads, but may still require communicating with the device to program therapy parameters or to upload diagnostic data. The placement of the device within the heart complicates the ability to communicate with device, and the size requirements of the device places restrictions on the energy available for these communications. The present inventors have recognized a need for improved communication with implanted leadless pacemakers.

### OVERVIEW

This document relates generally to systems, devices, and methods that provide electrical therapy to the heart or other structure of a patient or subject. In particular it relates to leadless implantable medical devices that provide electrical pacing therapy.

A device example can include a hermetically sealed housing including a cylindrical body, a first surface at a first capped end of the cylindrical body, and a second surface at a second capped end of the cylindrical body. A first electrode can be located at the first capped end and a second electrode can be located on the second surface. The first and second electrodes include conductive portions configured for contacting one or both of tissue and fluid, and wherein the cylindrical body includes a length and the conductive portions of the first and second electrodes are separated substantially by the length of the cylindrical body. The device example also includes a therapy circuit configured to deliver electrical cardiac stimulating energy using the first and second electrodes, and a telemetry circuit configured to communicate with a second separate device.

This section is intended to provide an overview of subject matter of the present patent application. It is not intended to provide an exclusive or exhaustive explanation of the disclosure. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, the various examples discussed in the present document.

FIG. 1 shows an example of a leadless implantable pacemaker.

FIG. 2 illustrates portions of another example of a leadless implantable medical device.

FIG. 3 shows a block diagram of portions of an example of an electronics unit for a leadless implantable medical device.

FIG. 4 illustrates portions of still another example of a leadless implantable medical device.

FIG. 5 illustrates portions of still another example of a leadless implantable medical device.

FIG. 6 illustrates portions of still other example of a leadless implantable medical device.

FIGS. 7A and 7B illustrate portions of still another example of a leadless implantable medical device.

FIG. 8 shows an example of a method of forming a leadless implantable medical device.

FIG. 9 illustrates portions of still another example of a leadless implantable medical device.

FIG. 10 illustrates portions of still another example of a leadless implantable medical device.

### DETAILED DESCRIPTION

An ambulatory medical device may include one or more of the features, structures, methods, or combinations thereof described herein. For example, an ambulatory cardiac monitor or cardiac stimulator may be implemented to include one or more of the advantageous features or processes described below. It is intended that such a monitor, stimulator, or other implantable, partially implantable, or wearable device need not include all of the features described herein, but may be implemented to include selected features that provide for unique structures or functionality. Such a device may be implemented to provide a variety of therapeutic or diagnostic functions.

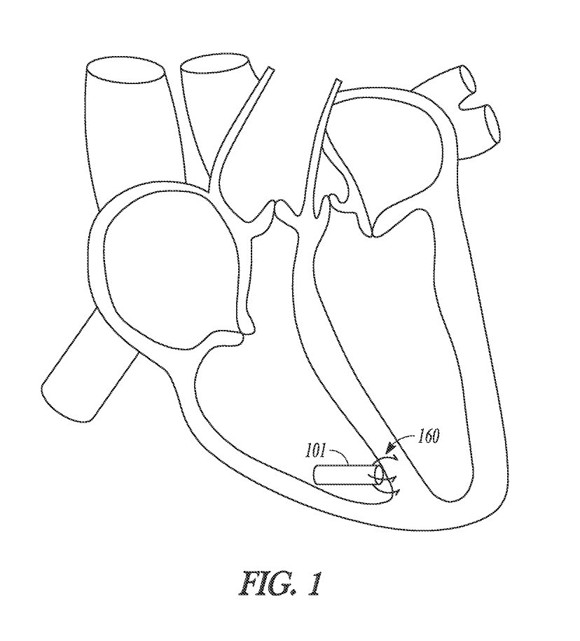

This document discusses systems, devices and methods for improved communication with implanted leadless medical devices. FIG. 1 shows an example of a leadless pacemaker 101. The leadless device is shown positioned at the endocardium within a ventricular chamber. The leadless device has a rod or bullet shape and includes electrodes arranged along the cylindrical portion of the housing. The leadless pacemaker 101 may include a fixation device 160 to fix or anchor the leadless pacemaker in contact with the myocardium. Some examples of a fixation device include one or more tines that extend radially from the housing, barbed tines, and a helix-shaped tine. An electronics unit can be contained within the housing.

One approach for communication with an implanted leadless medical device is to use conducted communication. In contrast to transmitted communication that involves a communication coil or antenna, conducted communication uses the body to transmit a communication signal. Information can be transferred between the implantable device and an external device by delivering electrical pulses using electrodes that are used for one or both of pacing and sensing of electrical therapy.

The stimulation pulses may be provided to the implantable device or sensed from the implantable by a separate external device (e.g., a device programmer). The separate external device may include two electrodes to contact the patient's skin to sense pulses from the implantable device and deliver pulse to the implantable device. The encoded stimulating or non-stimulating electrical energy may be provided to the implantable device or sensed from the implantable by a separate implantable leadless device (e.g., another leadless pacemaker or leadless cardioverter/defibrillator). Separate implantable devices may communicate to coordinate delivery of therapy (e.g., dual chamber pacing, bi-ventricular pacing, cardiac resynchronization therapy, and anti-tachycardia pacing therapy).

Electrodes arranged along the cylindrical portion of the housing can significantly limit the ability of the implanted leadless medical device to send information. This is because the electric field generated by an electrical pulse may remain localized near the device housing. Additionally, the ability of the device to detect external pulses can be limited by the spacing of the electrodes being too close together on the device housing. Conducted communication can be improved by changing the arrangement of the electrodes on the leadless device.

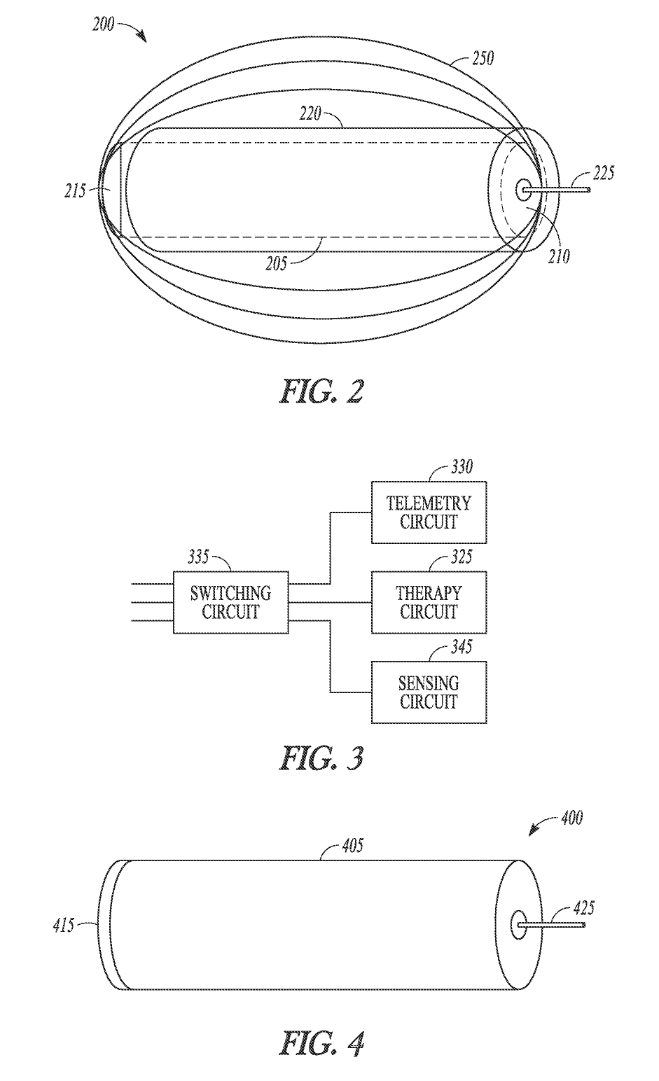

FIG. 2 illustrates portions of an example of a leadless implantable medical device 200. The device is used to provide electrical pacing therapy and to sense intrinsic cardiac activity. The device has a hermetically sealed housing that includes a cylindrical body 205, a first surface 210 at a first capped end of the cylindrical body 205, and a second surface 215 at a second capped end of the cylindrical body 205. A first electrode can be located at the first capped end and a second electrode can be located on the second surface 215. The first and second electrodes include conductive portions configured for contacting one or both of tissue and fluid. The cylindrical body 205 includes a length and the conductive portions of the first and second electrodes are separated substantially by the length of the cylindrical body 205.

In the example shown in FIG. 2, the cylindrical body 205 of the hermetically sealed housing is elongate (e.g., the length of the cylindrical body may be greater than the diameter of either of the first capped end or the second capped end). The second electrode can be located on (or incorporated into) the second surface 215 and the cylindrical body 205. In some examples, the second electrode is conductively connected to the hermetically sealed housing. The leadless implantable medical device 200 can include an electrically insulating coating 220 arranged over the elongate cylindrical body and extending substantially from a periphery of the first capped end to a periphery of the second capped end.

Some non-limiting examples of the insulating coating include silicone, parylene, urethane, acrylic, epoxy, and polytetrafluorethylene (PTFE). The electrically insulating coating 220 can serve to limit the effective surface area of the second electrode to the second surface 215 at the second capped end. This may result in improved radiation of the electric field 250 away from the device housing.

An electronics unit may be arranged within the housing of the device. FIG. 3 shows a block diagram of portions of an example of an electronics unit for the leadless implantable medical device 200. The electronics unit can include a therapy circuit 325 that delivers stimulating electrical energy using the first and second electrodes. The stimulating electrical energy may be electrical cardiac stimulation energy. In certain examples, the first electrode at the first capped end can be configured as a pacing cathode of the electrode pair and the second electrode at the second capped end can be configured as a pacing anode of the electrode pair. The electrically stimulating energy may be electrical neuron stimulating energy.

The therapy circuit 325 may provide electrical pacing therapy to treat bradycardia, In certain examples, the therapy circuit 325 provides anti-tachyarrhythmia pacing (ATP) therapy. The anti-tachyarrhythmia therapy by the leadless implantable medical device may be provided with one or both of anti-tachyarrhythmia cardioversion therapy and defibrillation therapy provided by a second separate device (e.g., a subcutaneously implantable cardioverter/defibrillator).

The electronics unit can include a telemetry circuit 330 that communicates with a second separate device. The second separate device can be an external device (e.g., an implantable device programmer or communicator) or another implantable device (e.g., an implantable cardioverter/defibrillator and the communication can be used to coordinate therapy). The telemetry circuit 330 may communicate information with the second separate device by the delivery of electrical energy to the first and second electrodes.

In some examples, the electrical energy for communication is non-stimulating electrical energy (sometimes referred to as sub-threshold stimulation), The electrical energy can be encoded pulses of electrical energy. The electrical energy can be made to be non-stimulating by reducing one or both of the magnitude of pulses and the width of the pulses so that the electrical pulses do not initiate a cardiac depolarization. In some examples, the communication is performed by embedding communication pulses within stimulating cardiac pacing pulses. In some examples, the cardiac pulses can be delivered during a refractory period that follows the onset of a cardiac action potential. The myocardium is not responsive to paced events during a refractory period even though the stimulation would normally initiate a cardiac event.

The electrodes are located at the ends of the leadless implantable medical device 200 and may perform double-duty as both pacing and communication electrodes. As shown in FIG. 3, the device may include a switching circuit 335 to switch between the therapy circuit 325 and the telemetry circuit 330 being applied to the electrodes. In some examples, there are separate electrodes for pacing and for conducted communication.

The ability of the leadless implantable medical device 200 to detect external pulses when implanted is improved by maximizing the separation between the electrodes. Additionally, any detrimental effects to the electric field due to the cylindrical housing being conductive may be mitigated by the electrically insulating coating. Further, FIG. 3 shows that the device may include a cardiac signal sensing circuit 345 to sense intrinsic signals using the electrodes. The increased separation may provide for better sensing of the signals. In certain examples, the separation between the electrodes may be thirty millimeters (30 mm). In certain examples, the separation between the electrodes may be within a range of 15 mm to 45 mm.

The arrangement of the electrodes can be improved further. In some examples, the first electrode is a pin electrode 225 located at the first capped end and arranged substantially orthogonal to the first capped end. The pin 225 can be placed into the endocardium of the patient or subject. The delivery of electrical pacing therapy energy from the cathode can be substantially at the end of the pin 225. This allows for the surface area of the cathode electrode to be small which can be advantageous for pacing, and allows provides additional separation between electrodes which can improve conducted communication.

FIG. 4 illustrates portions of another example of a leadless implantable medical device 400. The device includes a housing having an elongate cylindrical body 405, a first electrode incorporated into a pin 425 arranged at a first capped end of the housing, and a second electrode incorporated into a surface located at a. second capped end 415. Portions of the pin 425 and the surface of the second capped end 415 are conductive and contact tissue and fluid. The cylindrical housing is electrically insulating, The cylindrical housing can include a ceramic or plastic. As in the example of FIG. 2, the electrodes are located at the ends of the leadless implantable medical device 400 and may used for both pacing and communication. In some examples, the electrodes located at the ends of the implantable leadless device 400 are used for communication and a separate set of electrodes included on the housing for pacing therapy.

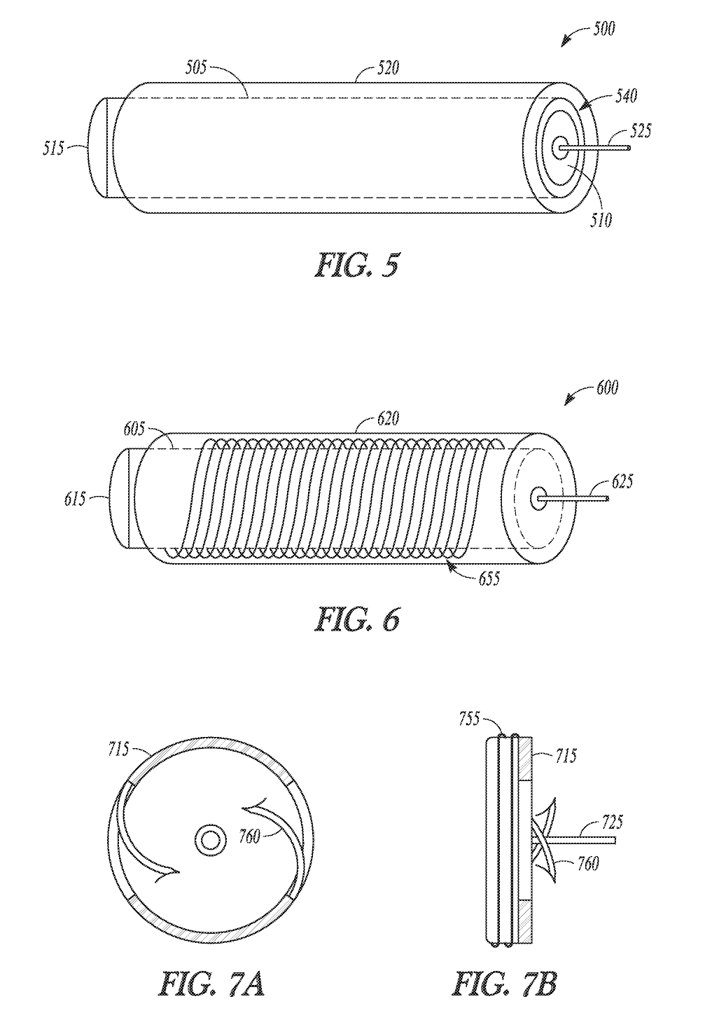

FIG. 5 illustrates portions of another example of a leadless implantable medical device 500. The device has a hermetically sealed housing that includes a cylindrical body 505, a first surface 510 at first capped end of the cylindrical body 505, a second surface 515 at a second capped end of the cylindrical body 505, and an electrically insulating coating 520 arranged over the cylindrical body 505. The device may include a first electrode incorporated into a pin 525 and a second electrode incorporated into the second surface 515. The device also includes a third electrode 540 arranged substantially at a periphery of the first surface of the hermetically sealed housing. The third electrode 540 may be substantially ring shaped. The second electrode may be configurable for both pacing, communication, and sensing, and the cardiac signal sensing circuit 345 of FIG. 3 may sense intrinsic electrical cardiac activity using the second electrode and the third electrode 540.

FIG. 9 illustrates portions of another example of a leadless implantable medical device 900. The device includes a first surface 910 at a first capped end of the cylindrical body 905, a second surface 915 at a second capped end of the cylindrical body 905. The device may include a first electrode incorporated into the first surface 910 and a second electrode incorporated into the second surface 915. The device includes a fixation mechanism 960. In the example of FIG. 9, the fixation mechanism includes tines that curl back from the first capped end. The tines may anchor the implantable device in the myocardium. The fixation mechanism can be coated with an electrically insulating material (e.g., silicone, parylene, urethane, acrylic, epoxy, or PTFE) to prevent the fixation mechanism from impacting conducted communication. In some examples, the entire device except for the electrodes may be covered with an electrically insulating material. The implantable device may further include a pin (not shown) that extends from the first capped and the pin may include an electrode.

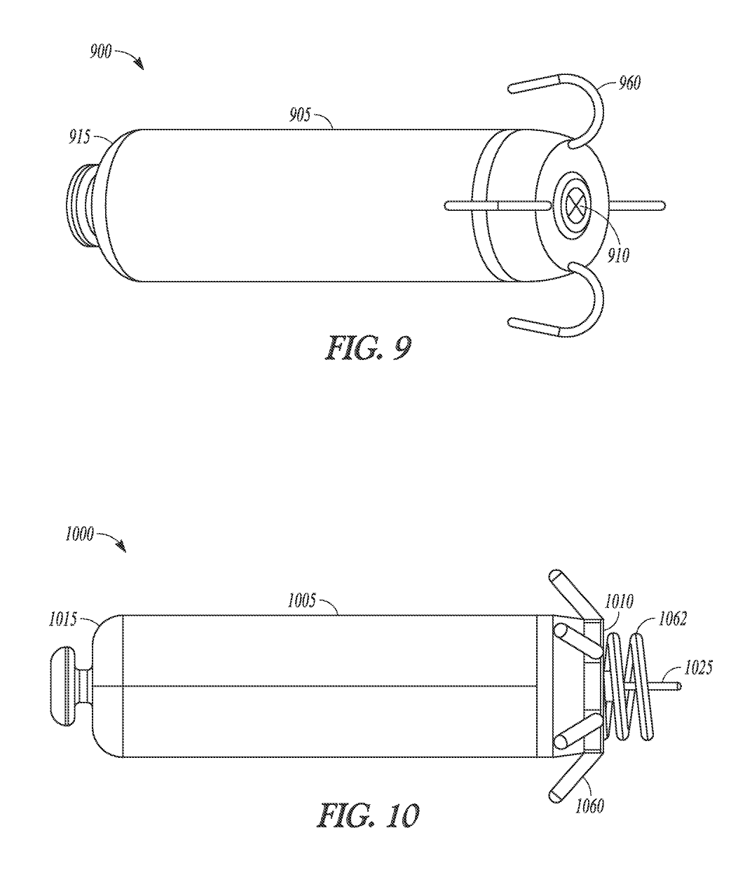

FIG. 10 illustrates portions of another example of a leadless implantable medical device 1000. The device includes a first surface 1010 at a first capped end of the cylindrical body 1005, a second surface 1015 at a second capped end of the cylindrical body 1005. The implantable device may include a first electrode incorporated into the first surface 1010 and a second electrode incorporated into the second surface 1015. The device includes a fixation mechanism 1060, 1062. In the example of FIG. 10, the fixation mechanism includes straight tines 1060 angled away from cylindrical body 1005 by less than ninety degrees. The tines may anchor the device in the myocardium. The fixation mechanism may include a helix 1062 with an anti-rotation feature. The fixation mechanism may include an electrically insulating coating to prevent the fixation mechanism from impacting conducted communication. As with the example of FIG. 9, the entire device except for the electrodes may be covered with an electrically insulating material. The implantable device may further include a pin (1025) that extends from the first capped and the pin may include an electrode.

The leadless implantable medical device may have a different mode of communication than the conducted communication described previously. In some examples, the leadless implantable medical device includes an antenna formed by an electrical conductor included within the electrically insulating coating. The telemetry circuit 330 of FIG. 3 communicates with the second separate device using the antenna.

FIG. 6 illustrates portions of another example of a leadless implantable medical device 600. The device includes a housing having an elongate cylindrical body 605, a first electrode incorporated into a pin 625 arranged at a first capped end of the housing, a second electrode incorporated into a surface located at a second capped end 615, and an electrically insulating coating 620 arranged over the elongate cylindrical body 605. The device also includes an inductive coil 655 formed by an electrical conductor contained within the electrically insulating coating. The inductive coil 655 may include windings of an insulated electrical conductor. The telemetry circuit of the device communicates with a second separate device using the inductive coil (e.g., using mutual inductance between inductive coils of the two devices).

In certain examples, the telemetry circuit of the device communicates with a second separate device using the inductive coil 655. In certain examples, the inductive coil 655 is used to transfer energy from the second separate device the leadless implantable medical device. The energy transferred may be used to charge a rechargeable battery of the leadless implantable medical device 600, or the energy transferred may be used to power the leadless implantable medical device 600. For example, the transferred energy can be applied to a storage capacitor included in the leadless implantable medical device 600 and the device is powered by the energy stored on the capacitor.

FIGS. 7A and 7B illustrate portions of another example of a leadless implantable medical device. The leadless implantable medical device includes a cylindrical body that is not elongate (e.g., the length of the cylindrical body of the hermetically sealed housing can be shorter than the diameter of one or both of the first and second surfaces) and has a disk-like shape or a button-like shape.

FIG. 7A shows a front view of the device, and FIG. 7B shows a side view of the device. In the example shown in the Figures, the device includes a housing having a short cylindrical body 705. The electrodes of the device may be configured for contact with the epicardium of the subject with a first electrode incorporated into a pin 725 arranged at a first surface of the housing, and a second electrode 715 incorporated into the first surface or a side surface. The device may include a fixation device 760 arranged to extend away from the first surface.

To facilitate communication with the device, the device can include an inductive coil formed or arranged substantially at the periphery of the cylindrical body 705. The device can include an electrically insulating coating over the cylindrical body 705 and the inductive coil can be formed within the electrically insulating coating. In some examples, the inductive coil is arranged within the housing and at the periphery of the housing. If the diameter of the example shown in FIGS. 7A and 7B has a larger diameter than the example shown in FIG. 6, the example of FIGS. 7A and 7B may have a better range of communication due to a larger amount of magnetic flux entering the inductive coil.

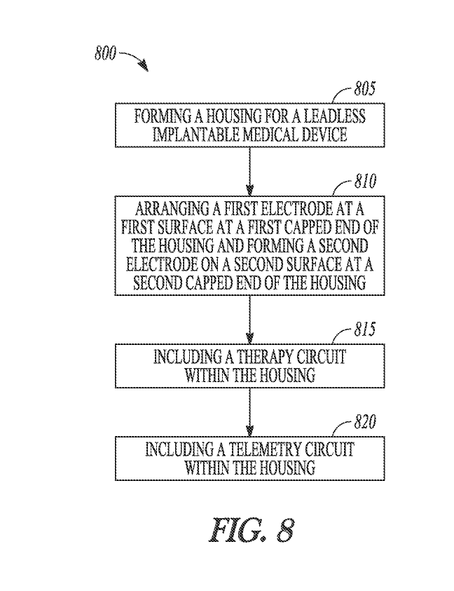

FIG. 8 shows an example of a method of forming a leadless implantable medical device. At block 805, a housing for the leadless implantable medical device is formed. The housing includes a cylindrical body, a first surface at a first capped end of the cylindrical body, and a second surface at a second capped end of the cylindrical body. In some examples the cylindrical body is elongate such as a short rod shape, and in other examples the cylindrical body is short and has a disk or button shape. An electrically insulating coating can be placed over the cylindrical body.

At block 810, a first electrode is arranged at the first capped end and a second electrode is formed on the second surface. In some examples, the electrodes are electrically isolated from the cylindrical body. In some examples, one of the electrodes is not electrically isolated from the cylindrical body, but an electrically insulating coating is used to limit the active area of the electrode to a capped end or a portion of a capped end. The first and second electrodes include conductive portions configured for contacting one or both of tissue and fluid. The cylindrical body includes a length, and the conductive portions of the first and second electrodes can be arranged so that they are separated substantially by the length of the cylindrical body. In some examples, the first electrode is a pin electrode arranged to be substantially orthogonal to the first capped end of the housing. None of the electrodes are included in implantable leads.

At block 815, a therapy circuit is included within the housing. The therapy circuit delivers electrical cardiac stimulating energy using the first and second electrodes. In some examples, a cardiac signal sensing circuit is included in the housing. A third electrode may be added to the device. The cardiac signal sensing circuit may use the second and third electrodes to sense the intrinsic signals.

At block 820, a telemetry circuit is included within the housing. The telemetry circuit communicates information with a second separate device. The telemetry circuit may be configurable for contact with the electrodes to communicate information using conducted communication. In some examples, an inductive coil or antenna is added to the device and the telemetry communicates using the inductive coil or antenna. In some examples, the inductive coil or antenna is added after a first insulating coating is applied to the cylindrical housing and a second insulating coating covers the inductive coil or antenna.

The several examples described herein do not include implantable leads. This allows the leadless implantable medical device to be small. The small size can complicate communication with the device. Different arrangements of the electrodes and different shapes of the device housing can improve different types of device communication. The examples have mostly been described in regard to leadless cardiac pacemakers. However, the examples can be equally useful in other types of implantable devices, such as in neuro-stimulation devices intended to treat pain, heart failure, hypertension, or epilepsy, and in implantable drug pumps. The examples can also be used in non-therapeutic devices such as implantable cardiac loop recorders and implantable heart failure monitors.

### ADDITIONAL NOTES AND EXAMPLES

The above detailed description includes references to the accompanying drawings, which form a part of the detailed description. The drawings show, by way of illustration, specific embodiments in which the disclosure can be practice These embodiments are also referred to herein as “examples.” In the event of inconsistent usages between this document and any documents incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim, Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

What s claimed is:

1. A leadless implantable medical device comprising:
a hermetically sealed housing including a cylindrical body, a first surface at a first capped end of the cylindrical body, and a second surface at a second capped end of the cylindrical body;
a first electrode located at the first capped end and a second electrode located on the second surface, wherein the first and second electrodes include conductive portions configured for contacting one or both of tissue and fluid, and wherein the cylindrical body includes a length and the conductive portions of the first and second electrodes are separated substantially by the length of the cylindrical body;
a therapy circuit configured to deliver stimulating electrical energy using the first and second electrodes; and
a telemetry circuit configured to communicate with a second separate device.

2. The leadless implantable medical device of claim 1, wherein the cylindrical body of the hermetically sealed housing is elongate, wherein the second electrode is located on the second surface of the elongate cylindrical body, and wherein the device further includes an electrically insulating coating arranged over the elongate cylindrical body and extending substantially from a periphery of the first capped end to a periphery of the second capped end.

3. The leadless implantable medical device of claim 2, wherein the therapy circuit is configured to deliver non-stimulating electrical energy to the first and second electrodes, and wherein the telemetry circuit is configured to communicate information with the second separate device by the delivery of electrical energy to the first and second electrodes.

4. The leadless implantable medical device of claim 2, wherein the second electrode is located on the second surface and is conductively connected to the hermetically sealed housing.

5. The leadless implantable medical device of claim 2, wherein the first electrode is a pin electrode located at the first capped end and arranged substantially orthogonal to the first capped end.

6. The leadless implantable medical device of claim 2, wherein the therapy circuit is configured to deliver the stimulating electrical energy using the first electrode as a cathode of an electrode pair and using the second electrode as the anode of the electrode pair.

7. The leadless implantable medical device of claim I, wherein the telemetry circuit is configured to communicate with the second separate device by detecting non-stimulating electrical energy at the first and second electrodes.

8. The leadless implantable medical device of claim 1, including an inductive coil, wherein the telemetry circuit is configured to communicate with the second separate device using the inductive coil.

9. The leadless implantable medical device of claim 8, wherein the cylindrical body of the hermetically sealed housing is an elongate cylindrical body, wherein the device further includes an electrically insulating coating arranged over the elongate cylindrical body and extending substantially from a periphery of the first surface to a periphery of the second surface, and wherein the inductive coil is formed by an electrical conductor contained within the electrically insulating coating.

10. The leadless implantable medical device of claim 8, wherein the length of the cylindrical body of the hermetically sealed housing is shorter than a diameter of one or both of the first and second surfaces and has a disk-like shape, wherein the inductive coil is formed substantially at a periphery of the cylindrical body.

11. The leadless implantable medical device of claim 1, including an electrically insulating coating arranged over the elongate cylindrical body and extending substantially from a periphery of the first capped end to a periphery of the second capped end, and an antenna formed by an electrical conductor included within the electrically insulating coating, and wherein the telemetry circuit communicates with the second separate device using the antenna.

12. The leadless implantable medical device of claim 1, including:
a third electrode arranged substantially at a periphery of the first surface of the hermetically sealed housing; and
a cardiac signal sensing circuit configured to sense intrinsic electrical cardiac activity using the second and third electrodes.

13. The leadless implantable medical device of claim 1, including a fixation mechanism wherein the fixation mechanism includes an electrically insulating material.

14. A method comprising:
forming a housing for a leadless implantable medical device, wherein the housing includes a cylindrical body, a first surface at a first capped end of the cylindrical body, and a second surface at a second capped end of the cylindrical body;
arranging a first electrode at the first capped end and forming a second electrode on the second surface, wherein the first and second electrodes include conductive portions configured for contacting one or both of tissue and fluid, wherein the cylindrical body includes a length, and wherein the conductive portions of the first and second electrodes are arranged so that they are separated substantially by the length of the cylindrical body;
including a therapy circuit within the housing, wherein the therapy circuit is configured to deliver electrical cardiac stimulating energy using the first and second electrodes; and
including a telemetry circuit within the housing, wherein the telemetry circuit is configured to communicate information with a second separate device.

15. The method of claim 14, wherein forming a housing includes forming a housing that includes an elongate cylindrical body,
wherein the method further includes arranging an electrically insulating coating over the elongate cylindrical body and extending the insulating coating substantially from a periphery of the first capped end to a periphery of the second capped end,
wherein the therapy circuit is configured to deliver electrical energy to the first and second electrodes, and
wherein the telemetry circuit is configured to communicate with the second separate device by the delivery of electrical energy to the first and second electrodes.

16. The method of claim 14, wherein forming a housing includes forming a housing that includes an elongate cylindrical body,
wherein the method further includes arranging an electrically insulating coating over the elongate cylindrical body and extending the insulating coating substantially from a periphery of the first capped end to a periphery of the second capped end; and forming an inductive coil within the electrically insulating coating,
wherein the telemetry circuit is configured to communicate with the second separate device using the inductive coil.

17. The method of claim 14, wherein arranging a first electrode includes locating a pin electrode at the first capped end and arranging the pin electrode substantially orthogonal to the first capped end.

18. The method of claim 14, including:
forming a third electrode arranged substantially at a periphery of the first surface of the housing; and
including a cardiac signal sensing circuit within the housing, wherein the cardiac signal sensing circuit is configured to sense intrinsic electrical cardiac activity using the second and third electrodes.

19. The method of claim 14, wherein forming a housing includes forming the housing to have a shape that is substantially disk-like so that a length of the cylindrical body of the housing is shorter than a diameter of one or both of the first and second surfaces, wherein the method further includes forming an inductive coil that is substantially arranged at a periphery of the cylindrical body, and wherein the telemetry circuit is configured to communicate with the second separate device using the inductive coil.

20. The method of claim 14, wherein forming a housing includes forming a housing that includes an elongate cylindrical body,
wherein the method further includes arranging an electrically insulating coating over the elongate cylindrical body and extending the insulating coating substantially from a periphery of the first capped end to a periphery of the second capped end; and
forming an antenna within the electrically insulating coating, wherein the telemetry circuit is configured to communicate with the second separate device using the antenna.
